(19) United States
(12) Patent Application Publication
(10) Pub. No.: US 2012/0190748 A1
(43) Pub. Date: Jul. 26, 2012

(54) GREATER UTILITY WITH THYROID HORMONE

(76) Inventor: Haren Treasurer, Toronto (CA)

(21) Appl. No.: 13/388,955

(22) PCT Filed: Aug. 3, 2010
(86) PCT No.: PCT/IB2010/002229

§ 371 Date: Apr. 16, 2012

Related U.S. Application Data
(60) Provisional application No. 61/231,151, filed on Aug. 4, 2009.

(51) Int. Cl. A61K 31/198; A61P 5/14; A61M 5/142; C07C 229/36

(52) U.S. Cl. 514/567; 562/444; 604/151; 604/500

(57) ABSTRACT

Formulations and delivery systems that include a thyroid hormone active agent consisting essentially of T4 should reduce fluctuations in delivery of thyroid hormone.

### CROSS-REFERENCE TO RELATED PATENT APPLICATIONS

This application claims priority from U.S. provisional application 61/231,151, filed Aug. 4, 2009, which is incorporated herein by reference in its entirety.

### FIELD

The present application generally relates to the field of hormonal therapy and, more particularly, to thyroid hormone therapy.

### SUMMARY

One embodiment of the invention comprehends an infusion system comprising (a) a container containing an infusate consisting essentially of thyroxine and (b) a driving member configured to drive said infusate from the container in a continuous manner, at a constant but user adjustable rate.

Another embodiment provides a plurality of thyroxine oral forms, wherein each such form comprises a hormonal active agent that consists essentially of thyroxine and wherein an incremental difference between said forms in the amount of the thyroxine present is no greater than 0.0000000125179946172623 moles.

Pursuant to yet another embodiment of the invention, an oral dosage form is provided that comprises a controlled release matrix containing a hormone active agent that consists essentially of thyroxine.

### DRAWINGS

FIG. 1 schematically depicts a catheter for an infusion system.

FIG. 2 relates to the catheter implantation and pump positioning on a human body.

FIG. 3 schematically illustrates an external infusion system and its components.

FIG. 4A schematically depicts an implantable infusion system and its components.

FIG. 4B schematically illustrates a location of an implantable infusion system on a human body.

FIG. 5 schematically illustrates a cartridge, which may be used, for example, with an external or implantable infusion system.

FIG. 6 schematically depicts a box of cartridges.

### DETAILED DESCRIPTION

Unless otherwise specified “a” or “an” means to one or more.

The terms “thyroxine” and “T4” refer to 3,5,3′,5′ tetraiodo-L-thyronine, a salt of 3,5,3′,5′ tetraiodo-L-thyronine, a prodrug form of 3,5,3′,5′ tetraiodo-L-thyronine, a salt of the prodrug form of 3,5,3′,5′ tetraiodo-L-thyronine or any combination thereof.

In this description, the phrases ‘consisting essentially of thyroxine’ and ‘consisting essentially of T4’ qualify an object, such as a formulation, a composition, a delivery system, a pharmaceutical product or an active agent, that includes one or more of the following entities: 3,5,3′,5′ tetraiodo-L-thyronine, a salt of 3,5,3′,5′ tetraiodo-L-thyronine, a prodrug form of 3,5,3′,5′ tetraiodo-L-thyronine, or a salt of the prodrug form of 3,5,3′,5′ tetraiodo-L-thyronine, but that excludes, as best as possible, any of the following entities: T3, also known as 3,5,3′ triiodo-L-thyronine, analogues, such as structural, functional, substrate, and transition state analogues, of 3,5,3′,5′ tetraiodo-L-thyronine, and analogues, such as structural, functional, substrate, and transition state analogues, of 3,5,3′ triiodo-L-thyronine.

In this regard, “best as possible” means that the excluded entities either are absent from the object that consists essentially of thyroxine or are present in the amount that does not detract from the beneficial and distinctive features of the invention, such as achieving reduced fluctuations in thyroid hormone volume of distribution and metabolism and/or achieving optimal distribution of thyroid hormone effect.

Use here of each of the terms ‘T4’, “thyroxine” and ‘3,5,3′,5′ tetraiodo-L-thyronine’ encompasses all isotopic variants of 3,5,3′,5′ tetraiodo-L-thyronine.

The phrase ‘prodrug form of 3,5,3’,5′ tetraiodo-L-thyronine' refers to a molecule in which an atom or atoms, in one or more side chains of 3,5,3′,5′ tetraiodo-L-thyronine, is/are substituted or covalently bonded with another molecule/macromolecule (denoted here “the carrier molecule/macromolecule), which, when administered to an organism, is likely to be metabolized (converted by physiologic processes and interactions with exogenous or endogenous substances present within the organism) into 3,5,3′,5′ tetraiodo-L-thyronine free of its covalent bonding to the carrier molecule/macromolecule. Examples of appropriate prodrugs include but are not limited to Thyroxinyldimethylphosphinate described in U.S. Pat. No. 6,627,660; peptide bound T4 described in U.S. Pat. No. 7,163,918; T4 bound by thyroglobulin/thyroid binding globulin/transthyretin/albumin, and T4 bound by polypeptide/peptide segments of thyroglobulin/thyroid binding globulin/transthyretin/albumin.

Thyroid Hormone Therapy

The primarily active forms of thyroid hormone within the body are 3,5,3′,5′ tetraiodo-L-thyronine (T4 or L-thyroxine) and 3,5,3′ triiodo-L-thyronine (T3). T4 and T3 are stored within the thyroid gland—one of the largest endocrine organs—as part of Thyroglobulin (Tg) protein molecules at a molar ratio of approximately 15:1 (18:1 by weight) (Kronenberg, 2008, ch 10; for citations see REFERENCES section, infra).

They are secreted from the gland at a ratio (by weight) of roughly 13:1 (Kronenberg, 2008, ch. 10). The thyroid gland has an extremely rich vasculature, receiving 4 to 6 ml/min/g of blood flow, setting itself far above almost any organ in the body (the kidney only receives 3 ml/min/g) (id.). As compared with other endocrine organs, the secretion of thyroid hormone by the thyroid gland is relatively constant.

Quantification of the exact ratio of T4 to T3 within thyroglobulin and the exact ratio secreted is difficult due to the likelihood of iodine atoms to be hydrolyzed from T4 and T3 during analysis, and thus the exact ratio of T4 to T3 is unknown and likely to be even greater. The ratio of T4 to T3 rises with iodine availability, and with the widespread iodination of foodstuffs, the majority of the world's population likely functions without iodine limiting the ratio (id.).

Both T4 and T3 are unstable molecules due to the likelihood of their iodine atoms to be removed by hydrolysis, though T3 is more unstable. Once secreted, thyroid hormone is tightly bound within the blood stream by Thyroid Binding Globulin, Transthyretin and Albumin—minimizing hydrolysis. T4 is bound roughly 10 times more tightly than T3 (99.96% vs 99.6%). (Kronenberg, 2008, ch. 10; Katzung, 1998, ch. 38). Due to protein binding, the biological half-life of T3 is extended to approximately 1 day, and the biological half-life of T4 is extended to approximately 6 to 7 days, giving T4 one of the longest biological half-lives of any hormone in the human body (Katzung, 1998, ch. 38). The secretion of T4 and T3 from the thyroid gland is stimulated by Thyroid Stimulating Hormone (TSH), the secretion of which—by anterior pituitary gland—is controlled by Thyrotropin-Releasing Hormone (TRH). TRH is expressed in many tissues throughout the body, however the parvocellular region of the paraventricular nucleus (PVN) of the hypothalamus is the source of the TRH that regulates TSH secretion. The PVN is influenced by various neural stimuli which in turn are influenced by the physical and psychological condition of the body, including body temperature, neural chemistry (such as the presence of a psychotic state), circadian and other pulsatile endocrine rhythms within the body, and severe physical and psychological stresses. The secretion of TSH and TRH is also inhibited by higher concentrations of T4 and or T3 in a negative feedback manner (Katzung, 1998, ch. 38).

At their site of action, both T3 and T4 cross the cell membrane. T4 is converted to produce reverse T3 (a relatively inactive isomer) and T3. T3 then enters the nucleus and binds the

Thyroid Hormone Receptor protein (TR), causing dissociation of a co-repressor (CoR) protein and allowing binding of co-activator (CoA) proteins, such as the Retinoid X Receptor protein (RXR). The T3-TR-RXR complex then influences the transcription of various genes differently than in their state while bound to the TR-CoR complex. The altered transcription alters the synthesis of various proteins (Kronenberg, 2008, ch. 10). The sum effect of these actions is unknown.

It is known that low concentrations of T4 and T3 produce symptoms that are termed hypothyroidism: cold intolerance, cool dry skin, brittle hair, decreased heart rate, decreased appetite/bowel movements, decreased GFR, stiffness, lethargy, slowing of cognition, hyperlipidemia, decreased hormone/vitamin/drug degradation and weight gain; the supplementation with exogenous T4 and T3 is intended to reduce the severity of these symptoms (Cummings, 2010, ch. 123).

High concentrations of T4 and T3 produce symptoms which is termed hyperthyroidism: heat intolerance, sweating, thin hair, increased heart rate, arrythmias, increased appetite/bowel movements, increased GFR, weakness, tremor, nervousness, hyperglycemia, increased hormone/vitamin/drug degredation and weight loss; the discontinuation of supplementation, ablation of the thyroid gland, or use agents which suppress T4 and T3 production is intended reduce the severity of these symptoms (Cummings, 2010, ch. 123).

The relationship between T4 and T3 concentrations and symptoms of thyroid disease may be complicated by the following factors:

1. some symptoms of hypothyroidism may occur with high or normal concentrations of T4 and/or T3, and that some symptoms of hyperthyroidism can occur with low or normal concentrations of T4 and/or T3;

2. weight gain may not be reliably corrected by normalizing T4 and/or T3 to a euthyroid levels;

3. normalization of high T4 and/or T3 concentrations may not reliably result in normalization of weight; rather weight gain often ensues despite euthyroid levels;

4. TSH may often be elevated with low concentrations of T4 and/or T3, and the contribution of TSH's trophic effect on the thyroid gland, the resulting increased endogenous thyroid hormone secretion, and the body's response to that changing amount has always been neglected when considering the cause of symptoms associated with a ‘hypothyroid state’;

5. TSH may often be low with elevated concentrations of T4 and/or T3, and the contribution of thyroid gland atrophy in response to low TSH levels, the resulting decreased endogenous thyroid hormone secretion, and the body's response to that changing amount has always been neglected when considering the cause of symptoms associated with a ‘hyperthyroid state’.

Synthetic T4 may be currently the treatment of choice for hypothyroidism and/or restoration of euthyroid TSH, T3 and T4 concentrations. T4 may be preferred over synthetic and other (bovine desiccated thyroid gland for example) preparations containing T4 and T3 in combination, or T3 alone, due to the shorter half life of T3 necessitating multiple daily doses, and greater risk of cardiotoxicity (palpitations, tachycardia, congestive heart failure and myocardial infarction)—originally thought to be due to greater activity of T3 relative to T4 (Katzung, 1998, ch. 38). Treatment protocols have recently been implemented with reduced incidence of cardiotoxic side effects with sustained release forms of T3 (Restorative Medicine, 2010), demonstrating that fluctuating T3 delivery is the cause cardiotoxicity associated with T3 administrations, not its increased activity relative to T4.

Based on the previous scientific studies (Brunova et al, 2003; Ross et al, 2003; Tigas et al, 2000; Dale et al, 2000; Pears et al, 1990; Saravanan et al, 2002; Wojewoda, 2005; Cohen, 2010) current means of treating hypothyroidism with only supplemental T4, may differ in some way from endogenous thyroid hormone delivery.

It has been proposed that treatment target euthyroid free T4 concentrations may be too low and target TSH concentrations may be too high. While some studies demonstrate positive correlations between TSH and body mass index (BMI) and/or waist circumference (WC), and weak to no inverse correlations between T4, T3, free T4, and/or free T3 concentrations and BMI or WC (Knudsen, 2005 and Michalaki, 2006), other studies find that no significant correlations exist (Kim, 2008 and Chomard, 1985).

Though it has also been proposed that T4 only therapy may be inadequate because T3 is present in endogenous production and secretion of thyroid hormone, and therefore must be present in suitable therapy, studies demonstrate that psychological/general and weight related deviation from the norm, is not corrected by the use of T3 only, or T3/T4 combination therapy (Valizadeh, 2009; Clyde, 2003; Escobar-Morreale, 2005; Siegmund, 2004; Regalbuto, 2007). Moreover, the ratio of T4 to T3 in endogenous production is greatly dominated by T4, and the secreted ratio varies widely, even within the same individual, with the ratio increasing as does the availability of iodine.

T4 has amongst the longest biological half life of any hormone in the human body (Kronenberg, 2008). Compared with other hormones that are under control from secretions of the hypothalamus, thyroid hormone is secreted with relative constancy (Kronenberg, 2008). The thyroid gland is one of the largest glands in the human body, has amongst the greatest perfusion/weight of any organ in the human body (allowing for homogenous distribution of thyroid hormone secretions throughout the body), and is the only gland designed to store its secretions in colloidal spheres in such large amount (Cummings, 2010, ch. 123). Thyroid hormone is primarily secreted as a prodrug (T4), which is deiodinated to a more active form (T3), also allowing for more homogenous distribution of thyroid hormone effect.

Current dosing guidelines primarily employed at present for thyroid hormone may be based on delivering the highest dose possible without suppression of TSH and/or consequences of a thyrotoxic state. Currently T4 is usually administered once daily.

The present inventor believes that current dosing causes fluctuations in thyroid hormone volume of distribution and metabolism, and suboptimal distribution of thyroid hormone effect throughout the body of a patient. The inventor believes that as the result of such disruptions TSH becomes suppressed (and along with it, any endogenous thyroid hormone secretion/delivery, if present) and the consequences of a thyrotoxic state occur. To avoid such disruptions, the present inventor developed thyroid hormone formulations and delivery systems that may allow reducing and/or minimizing these fluctuations, by reducing fluctuations in delivery of thyroid hormone (F). The present inventor also developed dosing/administering protocols for thyroid hormones that may allow reducing and/or minimizing these fluctuations and F.

Optimal distribution of thyroid hormone effect, in a mammal, may be defined as the distribution of thyroid hormone effect which occurs at steady state, and homeostatic equilibrium, when T4 is delivered continuously at a constant rate, homogenously across the cross sectional area of the junctions of the right atrium with the superior vena cava and inferior vena cava.

F can be defined as follows: ∫(|x−xₘₑₐₙ|)^{y}+W(∫(|y(x−xₘₑₐₙ₎^{y-1}|)^{z}), where ∫ is used to denote the integral during a specified period of time, such as the time between the beginning of one dose and the beginning of the next, or a 24 hour period, or a one week period, or a period of several years, where x is the rate, at a given point in time, of absorption/delivery of bioavailable thyroid hormone into the blood stream throughout the entire site of absorption, of the currently administered dose and previously administered dose(s). In this regard, xₘₑₐₙ is the mean of x at steady state or during the same specified period as the integral, y and z quantify the relative importance of the magnitude of the value to which they are applied, and W is a coefficient quantifying the relative importance of the value to which it applies. The present formulations and/or delivery systems may include a thyroid hormone active agent that consists essentially of T4; hence, excluding T3. The present inventor believes the presence of T3 to be undesirable because it may prevent optimally achieving reduced fluctuations (as compared with T4 alone) in thyroid hormone volume of distribution and metabolism, and because it may prevent optimal distribution of thyroid hormone effect, due to the short half life of T3. The present inventor believes that using formulations and/or delivery systems with a thyroid hormone agent consisting essentially of T4 should allow for a more accurate reproduction of thyroid hormone endogenous production in exogenous form, compared to conventional current therapy.

The present formulations and delivery systems may include a thyroid hormone active agent that consists essentially of T4, and therefore, exclude analogues, such as structural, functional, substrate, and transition state analogues of T3 and T4. The present inventor believes that the presence of such T3 and T4 analogues may be undesirable because of the uncertain reactivity/effects of these analogues, and the minimal to nil presence these analogues have in endogenous secretion. The present inventor believes that using present formulations and/or delivery systems with a thyroid hormone agent consisting essentially of T4 may allow achieving a more accurate reproduction of thyroid hormone endogenous production in exogenous form compared to therapy with T3 and T4 analogues.

Implicit in previous studies (Rangan, 2006; Bauhofer, 1976; Grebe, 1997) may be that reduced fluctuation in delivery of thyroxine results in increased TSH, and decreased T4, T3, free T4 and free T3 concentrations, for a give dose per unit time of thyroxine. The present inventor believes that using present formulations and/or delivery systems with a thyroid hormone agent consisting essentially of T4 may allow achieving greater TSH and lesser T4, T3, free T4, and free T3 concentrations, and therefore increased thyroid hormone volume of distribution and/or metabolism, for a given dose per unit time of thyroxine, compared to conventional current therapy.

Given the inventor's understanding of thyroid hormone function, and the diffuse constellation of symptoms and physiologic effects associated with hyperthyroid and hypothyroid states, the inventor believes that optimal physiology may be reduced, and many diseases may be potentiated, by fluctuation in thyroid hormone volume of distribution and metabolism and, therefore, by F as well.

The present formulations and delivery systems may be used for treating a condition for which a thyroid hormone therapy may be useful. The present formulations may be administered to a subject, which may be a vertebrate. In many embodiments, the subject may be a warm-blooded animal, such as a mammal. In a preferred embodiment, the subject may be a human being. The present delivery system may be used for administering a thyroid hormone active agent consisting essentially to a subject, which may be a vertebrate. In many embodiments, the subject may be a warm-blooded animal, such as a mammal. In a preferred embodiment, the subject may be a human being.

Applications of the present invention include treating thyroid disorders such as hypothyroidism, and hyperthyroidism. The applications of the present formulations and/or delivery systems also may include reducing body fat percentage, increasing lean mass (including body water and blood content), increasing tissue perfusion, improving the degree to which thyroid hormone effect is optimally distributed, and one or more benefits thereof, ultimately fortifying the physiology of a vertebrate organism, making it more resilient to and ameliorating the effects of: infectious, traumatic, genetic and polygenic multifactorial diseases, such as obesity, diabetes, hypertension, hyperlipidemia, hypercholesterolemia, hypertriglyceridemia, atherosclerosis, atopy, asthma, integumentary diseases, cardiovascular diseases, pulmonary diseases, gastrointestinal diseases, musculoskeletal diseases, neurologic/endocannabinoid diseases, immunologic diseases, nephrologic diseases, endocrine diseases, and genitourinary diseases.

Oral Forms

The present inventor developed an oral dosage formulation for thyroxine, which may reduce/minimize fluctuation in delivery of T4 into the bloodstream, when administered to a subject, such as a mammal, which may be a human. The oral dosage formulation disclosed in this section comprise a thyroid hormone active agent that consists essentially of thyroxine, which means that the thyroid hormone active agent is 3,5,3′,5′ tetraiodo-L-thyronine, a salt of 3,5,3′,5′ tetraiodo-L-thyronine, a prodrug form of 3,5,3′,5′ tetraiodo-L-thyronine, a salt of the prodrug form of 3,5,3′,5′ tetraiodo-L-thyronine, or any combination thereof and excludes, as best as possible: T3 also known as 3,5,3′ triiodo-L-thyronine; analogues, such as structural, functional, substrate, and transition state analogues of 3,5,3′,5′ tetraiodo-L-thyronine, and analogues of 3,5,3′ triiodo-L-thyronine, such as structural, functional, substrate, and transition state analogues of 3,5,3′ triiodo-L-thyronine.

In some embodiments, the oral dosage formulation may be a sustained, also known as prolonged, extended or timed, release formulation, which may provide a sustained release of thyroxine over a prolonged period of time, which may be at least 3 hours, at least 4 hours, at least 5 hours, at least 6 hours, at least 7 hours, at least 8 hours, at least 9 hours, at least 10 hours, at least 11 hours, at least 12 hours at least 13 hours, at least 14 hours, at least 15 hours, at least 16 hours, at least 17 hours, at least 18 hours, at least 19 hours, at least 20 hours, at least 21 hours, at least 22 hours, at least 23 hours or at least 24 hours.

The sustained release formulation may contain a controlled release matrix, which may contain a thyroid hormone active agent consisting essentially of thyroxine. In some embodiments, the controlled release matrix may be, for example, a polymer matrix. In some embodiments, the controlled release matrix may comprise a biodegradable material, such as a biodegradable polymer or a biodegradable porous silicon. In some embodiments, the controlled release matrix may comprise a porous material, such as a porous silicon or porous silica. In such a case, the hormone active agent may be contained in pores of the porous material.

“Sustained release”, “prolonged release”, and “prolonged action” are terms well known to those skilled in the art, see, e.g. Remington's at pages 1676 to 1693 Other related terms include “controlled release” and also U.S. Pat. Nos. 4,666,702 and 5,324,522.

Sustained release dosage forms, which may be used with the invention, include those described in the following patent documents: U.S. Pat No. 4,795,642, U.S. Pat. No. 4,851,232, U.S. Pat. No. 4,970,075, GB 2,219,206, U.S. Pat. No. 4,680,323, U.S. Pat. No. 4,357,469, U.S. Pat. No. 4,369,172, U.S. Pat. No. 4,389,393, U.S. Pat. No. 3,344,029, U.S. Pat. No. 4,012,498, U.S. Pat. No. 3,939,259, U.S. Pat. No. 3,065,143, U.S. Pat. No. 5,324,522, U.S. Pat No. 6,200,600, U.S. Pat No. 6,500,454, U.S. Pat No. 6,524,615, U.S. Pat. No. 7,083,808, U.S. Pat. No. 7,674,479, U.S. Pat. App. No. 20100136109.

The present oral forms may beused in treatment in place of conventional oral forms of T4, where ever they may be used.

The delivery of drugs as oral preparations in combination with a delivery vehicle that confers controlled release is known in the art. It may be desirable to formulate drugs in carriers that facilitate oral administration since this is less traumatic for subjects in need of drug treatment. In an effort to control drug release drug delivery vehicles have been designed that release an active agent in a controlled sustained manner resulting in a more constant supply rate of the drug into the blood stream. Means to deliver drugs in a sustained manner are known in the art and include slow release polymers that are formulated with a drug to control its release, see e.g. US patent publication no. 20100136109.

In some embodiments, the controlled release matrix may comprise drug delivery polymers, such as hydrophilic polymers, which generally are known in the art for allowing the controlled release of active agents either by diffusion out of the polymer matrix or by erosion of the polymer or a combination thereof. Polymers may be degradable or non-degradable but degradable may be preferred since they may degrade to smaller molecules that may be easily excreted. Examples of appropriate polymers include cellulose based polymers, such as hydroxypropylmethylcellulose, hydroxypropyl cellulose, methyl cellulose, and sodium carboxymethylcellulose; starch, including pre-gelatinised starch; polymethyacrylates and derivatives thereof, such as Eudragit RL and RS, polyvinyl pyrrolidone, polyvinyl alcohol, polyethelyene glycol, [poly (lactide-co-glycolide) and polyethylene oxide. Polymethyl methacrylate or hydroxypropylmethylcellulose may be preferred polymers.

In some embodiments, the polymer may be a non hydrophilic polymer, such as water insoluble ethyl derivatives (e.g., ethyl cellulose), microcrystalline cellulose (Avicel), and dicalcium phosphate.

The polymer morphology may also affect the release properties of the encapsulated drug and typically polymer matrices can be in the form of micro/nanoparticles, such as micro/nanospheres.

Examples of polymers used to obtain sustained drug release are also provided in WO99/22724, which describes the use of hydrophilic drug delivery polymers in the sustained release of venlafaxine; JP2006335771, which discloses the use of hydroxypropylmethylcellulose in the delivery of a number of medicines; WO0110443, which describes the use of hydroxypropylmethylcellulose in the sustained delivery of the anti-cancer agent camptothecin.

The present oral forms may contain at least 0.125 micrograms, or at least 0.25 micrograms or at least 0.5 micrograms or at least 1 microgram and no more than 1000 micrograms or any quantity within these ranges of T4 in an individual dose formulation, such as a tablet or a capsule.

The present oral dose forms may be administered at least once per day, or at least 2 times per day, or at least 3 times per day, or at least 4 times per day, or at least 5 times per day, or at least 6 times per day, or at least 7 times per day, or at least 8 times per day.

In some embodiments, the oral formulation may be a multiparticulate formulation. Such multiparticulate formulation may contain a thyroid hormone active agent consisting essentially of T4 in individual microparticulate units, which may be contained within a capsule. The multiparticulate formulation may comprise polyvinylpyrrolidone and optionally one or more additional components, such as microcrystalline cellulose, which may be Avicel; dicalcium phosphate; and lactose. In some embodiments, the multiparticulate may comprise polyvinylpyrrolidone and a mixture of two or more of the following: microcrystalline cellulose (e.g. Avicel), dicalcium phosphate, lactose. Polyvinylpyrrolidone may provided at between 0.5% w/w and 5% w/w; or 1-3% w/w or at about 1% w/w.

The polymer release matrix may also include excipients that can be added to one or more polymers to further modify drug release, drug stability or polymer degradation kinetics or combinations thereof. For example, basic salts may be added to control polymer degradation thereby altering drug release. In some embodiments, hydrophilic excipients may be added that accelerate drug release.

When administered, the medicament of the present invention is administered in pharmaceutically acceptable preparations. Such preparations may routinely contain pharmaceutically acceptable concentrations of salt, buffering agents, preservatives, compatible carriers and supplementary potentiating agents. The preferred route of administration is oral.

When administered, the formulation may be applied in pharmaceutically-acceptable amounts and in pharmaceutically-acceptable compositions. The term “pharmaceutically acceptable” means physiologically or toxicologically tolerable. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts may be prepared as alkaline metal or alkaline earth salts, such as sodium, potassium or calcium salts.

The formulation may contain a pharmaceutically-acceptable carrier. The phrse “pharmaceutically-acceptable carrier” is employed here to denote one or more compatible solid or liquid fillers, diluents, or encapsulating substances that are suitable for administration into a human. The term “carrier” refers to an organic or inorganic ingredient, natural or synthetic, with which the active ingredient is combined to facilitate the application. The components of the pharmaceutical compositions also are capable of being co-mingled with T4, and with each other, in a manner such that there is no interaction which would substantially impair the desired pharmaceutical efficacy.

The formulation may contain suitable buffering agents, including: acetic acid in a salt; citric acid in a salt; boric acid in a salt; and phosphoric acid in a salt. The formulation also may contain, optionally, suitable preservatives, such as: benzalkonium chloride; chlorobutanol and parabens.

The formulation may conveniently be presented in unit dosage form, such as a tablet or a capsule. and may be prepared by any of the methods well-known in the art of pharmacy. Such methods may include the step of bringing the active agent into association with a carrier which constitutes one or more accessory ingredients. In general, the formulation may be prepared by uniformly and intimately bringing the active compound, thyroxine into association with a liquid carrier, a finely divided solid carrier, or both, and then, if necessary, shaping the product.

Compositions suitable for oral administration may be presented as discrete units, such as capsules, pills, tablets, lozenges, gelcaps or caplets, each containing a predetermined amount of the active agent per unit weight. Other compositions include suspensions in aqueous liquids or non-aqueous liquids such as suspension, syrup, elixir or an emulsion suitable for oral administration, containing a predetermined amount of the active agent per unit weight. In some embodiments, the sustained release formulation may provide a sustained release for T4 may be up to 24 hours. Examples of suitable sustained release formulations may be found in U.S. Pat No. 5,324,522 and U.S. Publication 20100136109.

In some embodiments, the sustained released formulation comprising a thyroid hormone active agent consisting essentially of thyroxine may be achieved by using multilayered structures and chemicals described in U.S. Pat. Nos. 6,500,454, 7,083,808, and 7,674,479, or by using multicomparment structures and chemicals described in U.S. Pat. No. 7,470,435.

In some embodiments, the formulation may include a delayed delivery vehicle encapsulating the hormonal active agent in order to allow for doses which would otherwise be administered singly with a fixed interval between them, multiple times a day, to be administered simultaneously, less often; such encapsulation is described, for example, in U.S. Pat. No. 6,200,600.

The present inventor also believes that the reduced/minimized fluctuation of thyroid hormone may be achieved by using a greater variety of individual doses than currently available. Accordingly, a pharmaceutical product may comprise a plurality of individual thyroxine oral dosage forms, each of which comprising active agent consisting essentially of thyroxine, wherein the incremental difference in the amount, in moles, of thyroxine—here referring to thyroxine in the form of 3,5,3′,5′ tetraiodo-L-thyronine, a salt of 3,5,3′,5′ tetraiodo-L-thyronine, a prodrug form of 3,5,3′,5′ tetraiodo-L-thyronine, or a salt of the prodrug form of 3,5,3′,5′ tetraiodo-L-thyronine—between each two individual whole, unbroken, dosage forms in the product is greater than 0 but less than 0.0000000156474932715779 moles [equivalent to 12.5 μg of levothyroxine sodium], or greater than 0 but less than 0.0000000150215935407148 moles [equivalent to 12.0 μg of levothyroxine sodium], or greater than 0 but less than 0.0000000143956938098517 moles [equivalent to 11.5 μg of levothyroxine sodium], or greater than 0 but less than 0.0000000137697940789885 moles [equivalent to 11.0 μg of levothyroxine sodium], or greater than 0 but less than 0.0000000131438943481254 moles [equivalent to 10.5 μg of levothyroxine sodium], or greater than 0 but less than 0.0000000125179946172623 moles [equivalent to 10.0 μg of levothyroxine sodium], or greater than 0 but less than 0.0000000118920948863992 moles [equivalent to 9.5 μg of levothyroxine sodium], or greater than 0 but less than 0.0000000112661951555361 moles [equivalent to 9.0 μg of levothyroxine sodium], or greater than 0 but less than 0.000000010640295424673 moles [equivalent to 8.5 μg of levothyroxine sodium], or greater than 0 but less than 0.0000000100143956938099 moles [equivalent to 8.0 μg of levothyroxine sodium], or greater than 0 but less than 0.00000000938849596294674 moles [equivalent to 7.5 μg of levothyroxine sodium], or greater than 0 but less than 0.00000000876259623208362 moles [equivalent to 7.0 μg of levothyroxine sodium], or greater than 0 but less than 0.0000000081366965012205 moles [equivalent to 6.5 μg of levothyroxine sodium], or greater than 0 but less than 0.00000000751079677035739 moles [equivalent to 6.0 μg of levothyroxine sodium], or greater than 0 but less than 0.00000000688489703949427 moles [equivalent to 5.5 μg of levothyroxine sodium], or greater than 0 but less than 0.00000000625899730863116 moles [equivalent to 5.0 μg of levothyroxine sodium], or greater than 0 but less than 0.00000000563309757776804 moles [equivalent to 4.5 μg of levothyroxine sodium], or greater than 0 but less than 0.00000000500719784690493 moles [equivalent to 4.0 μg of levothyroxine sodium], or greater than 0 but less than 0.00000000438129811604181 moles [equivalent to 3.5 μg of levothyroxine sodium], or greater than 0 but less than 0.00000000375539838517869 moles [equivalent to 3.0 μg of levothyroxine sodium], or greater than 0 but less than 0.00000000312949865431558 moles [equivalent to 2.5 μg of levothyroxine sodium], or greater than 0 but less than 0.00000000250359892345246 moles [equivalent to 2.0 μg of levothyroxine sodium], or greater than 0 but less than 0.00000000187769919258935 moles [equivalent to 1.5 μg of levothyroxine sodium], or greater than 0 but less than 0.00000000125179946172623 moles [equivalent to 1.0 μg of levothyroxine sodium], or greater than 0 but less than 0.000000000625899730863116 moles [equivalent to 0.5 μg of levothyroxine sodium]. Each individual, whole, unbroken dosage form in the product may contain, in moles of thyroxine, as described above, no less than 0.000000000156474932715779 moles [equivalent to 0.125 μg of levothyroxine sodium] to no more than 0.00000125179946172623 moles [equivalent to 1000 μg of levothyroxine sodium], or no less than 0.000000000156474932715779 moles [equivalent to 0.125 μg of levothyroxine sodium] to no more than 0.00000100143956938099 moles [equivalent to 800 μg of levothyroxine sodium], or no less than 0.000000000156474932715779 moles [equivalent to 0.125 μg of levothyroxine sodium] to no more than 0.000000751079677035739 moles [equivalent to 600 μg of levothyroxine sodium], or no less than 0.000000000156474932715779 moles [equivalent to 0.125 μg of levothyroxine sodium] to no more than 0.000000625899730863116 moles [equivalent to 500 μg of levothyroxine sodium], or no less than 0.000000000156474932715779 moles [equivalent to 0.125 μg of levothyroxine sodium] to no more than 0.000000500719784690493 moles [equivalent to 400 μg of levothyroxine sodium], or no less than 0.000000000156474932715779 moles [equivalent to 0.125 μg of levothyroxine sodium] to no more than 0.000000375539838517869 moles [equivalent to 300 μg of levothyroxine sodium], or no less than 0.000000000156474932715779 moles [equivalent to 0.125 μg of levothyroxine sodium] to no more than 0.000000250359892345246 moles [equivalent to 200 μg of levothyroxine sodium], or no less than 0.000000000156474932715779 moles [equivalent to 0.125 μg of levothyroxine sodium] to no more than 0.000000187769919258935 moles [equivalent to 150 μg of levothyroxine sodium], or no less than 0.00000000125179946172623 moles [equivalent to 1 μg of levothyroxine sodium] to no more than 0.000000187769919258935 moles [equivalent to 150 μg f levothyroxine sodium], or no less than 0.00000000125179946172623 [equivalent to 1 μg of levothyroxine sodium] to no more than 0.000000156474932715779 moles [equivalent to 125 μg of levothyroxine sodium], or no less than 0.00000000125179946172623 moles [equivalent to 1 μg of levothyroxine sodium] to no more than 0.000000125179946172623 [equivalent to 100 μg of levothyroxine sodium].

Such a product may allow for a greater variety of delivered doses to more precisely and accurately achieve biological targets, allow for a more gradual titration when changing from a current total daily dose to a different target total daily dose, even if only administered once a day; most importantly such a product may facilitate multiple daily dosing regimes, comprising at least once per day, or at least 2 times per day, or at least 3 times per day, or at least 4 times per day, or at least 5 times per day, or at least 6 times per day, or at least 7 times per day, or at least 8 times per day, which may reduce fluctuation by reducing the maximum absolute deviation from the mean absorption rate. Multiple daily dosing regime may be facilitated by allowing for regimes involving multiple daily doses to achieve a greater variety of total/sum daily doses, while also allowing for a greater variety of dosing frequencies in achieving a given total/sum daily dose.

Besides the hormonal active agent, each individual oral dose form may include an orally acceptable excipient and/or additive.

Each individual oral form may be in a solid dosage form such as a tablet, pill, capsule, gelcap, caplet, lozenge, granule, or in containers, such as sachets or bottles, of powder, suspension, solution, emulsion, elixir, colloid, granules. Such forms may be prepared, for example, by mixing the thyroid hormone active agent with at least one additive or excipient, such as a starch or other additive. Suitable additives or excipients include sucrose, lactose, cellulose sugar, mannitol, maltitol, dextran, sorbitol, starch, agar, alginates, chitins, chitosans, pectins, tragacanth gum, gum arabic, gelatins, collagens, casein, albumin, synthetic or semi-synthetic polymers or glycerides, methyl cellulose, hydroxypropylmethyl-cellulose, and/or polyvinylpyrrolidone.

Optionally, oral dosage forms may contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Additionally, dyestuffs or pigments may be added for identification. Tablets and pills may be further treated with suitable coating materials known in the art.

In some embodiments, the individual oral dose forms may have the same composition as in commercially available, such as Levothyroxine sodium (T4), which is available under tradenames such as Eltroxin; Levothroid; Levoxine; Levoxyl; Novothyrox; Unithroid and Synthroid.

In some embodiments, the individual oral dosage form may comprise a sustained or controlled release formulation, such as the ones disclosed above.

In some embodiments, the individual oral dosage form may be comprised of an immediate release formulation, such as the ones disclosed in U.S. Pat. Nos. 7,101,569 and 6,555,581. In some embodiments, oral dosage forms may be packaged in a blister pack, such as has been done for levothyroxine sodium dosage forms sold under the company name of Goldshield, comprising a plurality of unit packaging regions, such that each region contains and encloses an individual thyroxine oral dose, comprising active agent consisting essentially of thyroxine, said pack comprising: a blister film sheet having unit package regions, wherein each unit package region consist of a cavity and a flange surrounding said cavity, each cavity being adapted to receive a unit dosage form, and a lidding sheet sealed to said flanges of the blister film sheet for enclosing a unit dosage form within each unit package region.

Each individual, whole, dosage form may not be intended to be broken by the administrator, as the breaking of such forms is not likely to produce parts of the whole with precisely and accurately known amounts of thyroxine, without the use of an analytic scale, resulting in increased fluctuation in delivery. If supplied to the administrator in a form which may be broken or separated into parts of the whole with precisely and accurately known amounts of thyroxine, without the use of an analytical scale, each such separate part of the whole may be considered a whole, unbroken, dosage form.

Infusion Systems

In some embodiments, a composition (infusate), which includes a hormonal active agent consisting essentially of thyroxine may be administered using an infusion system, which may include a container containing the infusate composition and a driving member or element, such as a pump, which may be configured to deliver continuously, the composition from the container. In many embodiments, the composition may be delivered at a constant, but user adjustable rate. At least one of the containers may contain in its inner volume the infusate containing a thyroid hormone active agent consisting essentially a thyroxine, which means that the thyroid hormone active agent is 3,5,3′,5′ tetraiodo-L-thyronine, a salt of 3,5,3′,5′ tetraiodo-L-thyronine, a prodrug form of 3,5,3′,5′ tetraiodo-L-thyronine, a salt of the prodrug form of 3,5,3′,5′ tetraiodo-L-thyronine, or any combination thereof. The infusate composition excludes, as best as possible, T3 also known as 3,5,3′ triiodo-L-thyronine; analogues, such as structural, functional, substrate, and transition state analogues, of 3,5,3′,5′ tetraiodo-L-thyronine, and analogues of 3,5,3′ triiodo-L-thyronine.

In some embodiments, the infusion system may include a pump assembly, which may include the pump per se and one or more containers, such as vials or cartridges. In some embodiments, at least one of the containers may also contain a maintenance solution, which may comprise one or more elements, such as anticoagulants, for example heparin, proteolytic enzymes, for example streptokinase, and a solution of sodium chloride, which may be, for example, at 0.9% (w/v) concentration in water. The pump assembly may also include one or more ports through which the composition may be delivered to a patient. In some embodiments, the infusion system may also include a tube or a catheter, which may be a plastic tube or catheter, through which the composition may be infused to a blood vessel, such as a vein, of the patient. One end or tip (distal) of such a catheter or tube may be introduced in the blood vessel of the patient's body, while the other end or tip (proximal) may be connected to at least one of the ports of the pump assembly. The tube or catheter may comprise, for example, a plastic material, such as polyurethane or silicone. In some embodiments, the catheter may be a single port catheter, which means that it has only one point of attachment at each of its ends. Yet in some embodiments, the catheter may be a multiport catheter, which means a catheter having more than one point of attachment at at least one of its ends.

One example of an external infusion system may be based on Medtronic's MiniMed Paradigm REAL-Time Revel System for diabetics, which is used for insulin infusion. The commercial Medtronic's system may be modified so that it does not have a glucose sensor. Additional modifications/adjustments may be made with respect to the pump, the catheter's length, design and placement, as well as in the number of phases in the cartridge syringe. The most significant difference with respect to the Medtronic's system may be that the infusate comprises a hormonal active agent that consists essentially of T4.

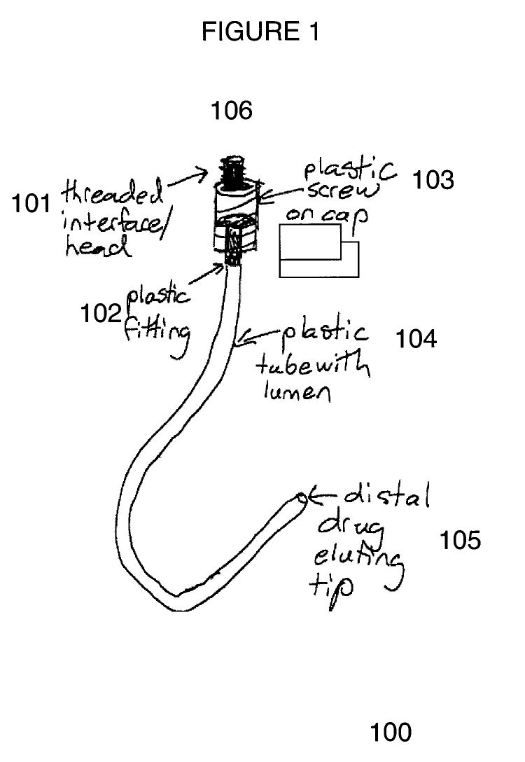

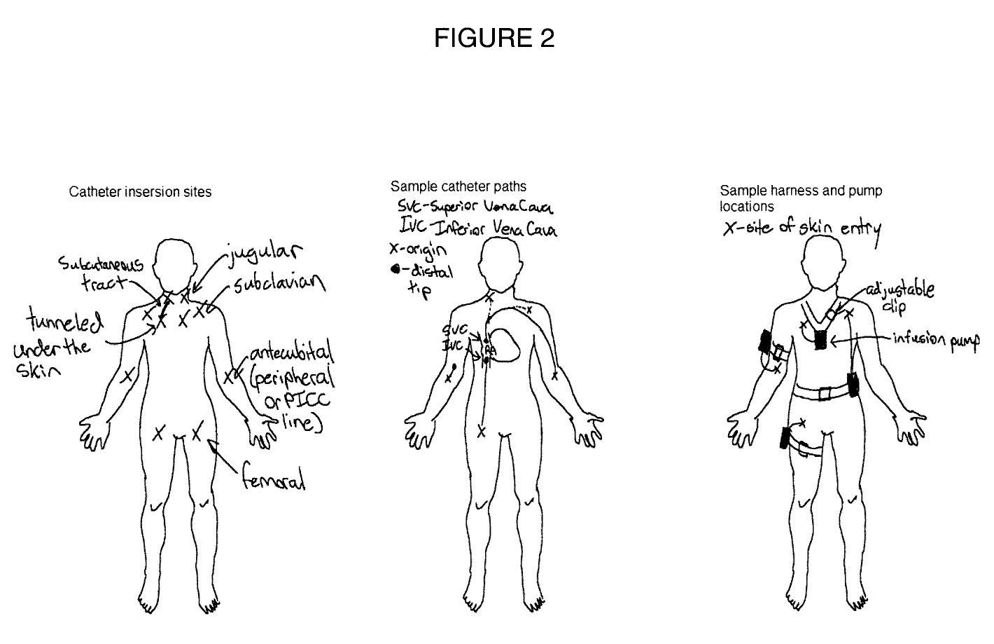

The catheter of the infusions system may include a sterile, plastic tube, such as a polyurethane or silicone tube. The silicone tube may be preferred. The catheter may include a single proximal port, a lumen, which may be sufficiently narrow to minimize dead space without obstructing the infusate flow, and a single distal drug eluting tip. The catheter may have a length available up to 1,5 meters or up to 1 meter. The catheter may be provided in a sterile packaging, which may also include devices required for insertion by cannula-over-needle or Seldinger technique. Such devises may include a needle, a trocar, a syringe and/or a guidewire. Upon the insertion, the catheter may enter the skin. The catheter may be tunneled through the subcutaneous tissue to the site of entry into a blood vessel, such as a vein. The tunneling of the catheter may done in a manner similar to a Hickman line. Upon completion of the tunneling, the catheter may enter a blood vessel, which may be a vein, such as an antecubital vein, a femoral vein, a jugular vein, a subclavian vein or a brachiocephalic vein. FIG. 2 (left diagram) schematically illustrates various catheter insertion sites on the body of a patient. Then the catheter may be advanced with its drug eluting tip being placed in one of the following final positions: 1. within the vein distal to the site of entry, 2. just superior to the junction of the superior vena cava to the right atrium, 3. just inferior to the junction of the inferior vena cava to the right atrium, 4. marginally within the right atrium. In some embodiments, the catheter, used in conjunction with the external infusion system, may be inserted into the alimentary canal by means of nasogastric passage, gastric fistula, ileal fistula and jejunal fistula. FIG. 2 (central diagram) schematically illustrates sample catheter paths in a human body after insertion. Positioning of the catheter and its distal tip may be confirmed with X-ray as well as by pressure transduction and analysis of the pressure waveform of the environment, where the catheter's tip is situated. In some cases, the proximal tip of the catheter may be covered with a port, which may include a replaceable head. Such a port may allow interfacing the catheter with a pump assembly, which may be outside of the skin. In some cases, the proximal tip may be covered with a port which may include a replaceable cap, which may comprise a seal, such as a plastic seal, a silicone seal or a plastic/rubber seal, which may remain beneath the skin, similar to Port-a-Cath or MediPort. In such a case, a secondary catheter may be included, with a hollow bore needle, and adhesive bandage, at its distal end which may pass through the skin and through the seal. The secondary catheter may be attached to the pump proximally via a connector, which may be a threaded connector. The catheter may have an affixed clip, which may be located just distal to its proximal tip. Such a clip may be sutured into place, held in place by, for example, an adhesive dressing, or hooked onto elastic mesh covering the area of insertion. FIG. 1 schematically depicts a modified catheter 100, which includes a plastic tube with lumen 104 and distal and proximal ends 105 and 106, respectively. The proximal end 106 of the catheter 100 includes an assembly comprising fitting 102, screw 103 on top of the fitting 102 and threaded interfacial head 101 on top of the screw 102. The interfacial head 101 may be used for connecting the catheter to the pump assembly directly or through an additional catheter.

The modified pump assembly may include a pump, which may be, for example, a battery powered, variable flow rate, pump. In some embodiments, the pump may be a motor and screw driven pump. The pump may be contained in a case. Such a case may be a plastic or a metal case. In some embodiments, a titanium case may be preferred. The pump assembly may be located externally on the body. The pump assembly may include a port for an electrical power connector. Such a port may be used to provide electricity and if needed, to charge the battery and drive the pump. The external infusion system may also include a transformer that may be plugged into an electrical outlet and connected to the electrical power port on the pump assembly. The pump assembly may include an attachment mechanism or tool. Such a mechanism or tool may be, for example, a harness. The attachment mechanism or tool may allow affixing or suspending the pump assembly from a part of the body, which may be for example, an arm, a leg, the thorax, or the waist. FIG. 2 (right diagram) schematically depicts exemplary positions for the pump assembly on the body of the patient. The pump assembly may include one or more sockets configured to be inserted with a container, such as a cartridge or vial. The socket may be equipped with one or more sensors configured to detect a fluid level in the inserted container . Such a sensor may be, for example, an optical or an ultrasonic sensor. The container, to be inserted, may contain the infusate or a maintenance solution. The socket for the container's insertion may include an attachment tool, such as a cover, which may secure the container in place. In certain embodiments, the cover may be opaque and thereby protect the container from the light. The pump assembly includes a pump per se. In case of multiple containers in the pump assembly, the pump may automatically switch its infusate supply between the containers as they become depleted. Such an option may allow for replacement of a depleted container without interrupting the infusion. The infusion system may include also a computer and one or more battery compartments. In many embodiments, the pump per se, the computer and/or the battery compartments may be thermally isolated within the pump assembly from a compartment that contains the infusate container(s) to minimize heat transfer into the container(s). The thermal isolation may involve, for example, separating the pump per se, the computer and/or the battery compartments from the container compartment with a vacuum sealed, plastic lined layer.

The pump assembly may include a display, which may be an lcd display. In some embodiments, the display may be a detachable display. In some embodiments, the display may have its own battery, which may be a rechargeable battery. Preferably, the display's battery is separate from the one powering the pump per se. The display may have a wire or wireless transmitter-receiver which may allow it to communicate with the system's computer. The pump assembly may include a port into which the display's interface may be attached, supplying power to charge the display's battery. The display may act as a user's interface with the system. The display may be used, for example, to operate the system, which may involve performing calibration, defining preferences and flow rate, as well as to display the system's and/or patient's parameters and information, such as pressure waveform, pump diagnostic information, amount of the infusate remaining in each cartridge, and displaying battery life remaining

The system may include a computer, which may be a computer built in the pump assembly. The computer may be used, for example, to control pump activity based on user settings; control the diverter valve; monitor the amount of the infusate remaining; monitor, record and/or analyze the pressure wave form; monitor for the presence of air within the infusion circuit, monitor for infusion circuit obstruction, monitor the battery life remaining; communicate with the display and/or to produce signals, which may, for example, signal the battery depletion and the infusate's container depletion, presence of air within the infusion circuit, and the location of the catheter's distal tip. The signals produced by the computer may be audible signals. For producing the audible signals, the system may include a speaker, which may integrated with the computer.

The external infusion system may also include an infusate conduit, which may be replaceable. In some embodiments, the conduit may be a ‘Y’ shaped infusate conduit. The conduit may be made, for example, of plastic or metal tubing. The ‘Y’ shaped infusate conduit may have two proximal ends (with respect to the conduit the term “proximal” end may mean being close to the infusate container(s) and the term “distal” may mean being away from them). Each of the proximal ends of the conduit may include a connector allowing for the infusate container to be plugged in. The distal end of the conduit may have a filter. Such a filter may be used to remove from the infusate undesirable components, such as precipitates. The distal end of the conduit may include an interface port for the proximal end of the primary catheter. The ‘Y’ shaped conduit may include a diverter valve, which may be positioned at the point of convergence of its proximal limbs.

In some embodiments, the conduit may include one or more of the following devices from proximal to distal: 1. a container connector, which may be a female connector. The container connector may be made of plastic. 2. a first sensor, which may be an air-in-line and/or a flow sensor. The sensor may be, for example, an ultrasonic or an optical sensor. 3. a first pressure transducer, 4. a diverter calve, which may be positioned at the point of convergence of its proximal limbs, 5. a second pressure transducer, 6. a second sensor, which may be an air-in-line and/or a flow sensor. The sensor may be, for example, an ultrasonic or an optical sensor. 7. a conduit branch connected to a port configured to remove air/or and addition of an infusate. The port may be a plastic port. The air removal and/or infusate addition may be done using a needle or a syringe. 8. flow shut off valve, 9. a combined pressure transducer and sensor, such as an air-in-line and/or a flow sensor. 10. a catheter connector, which may include a filter, which may be built into the connector. The catheter connector may be female a threaded connector.

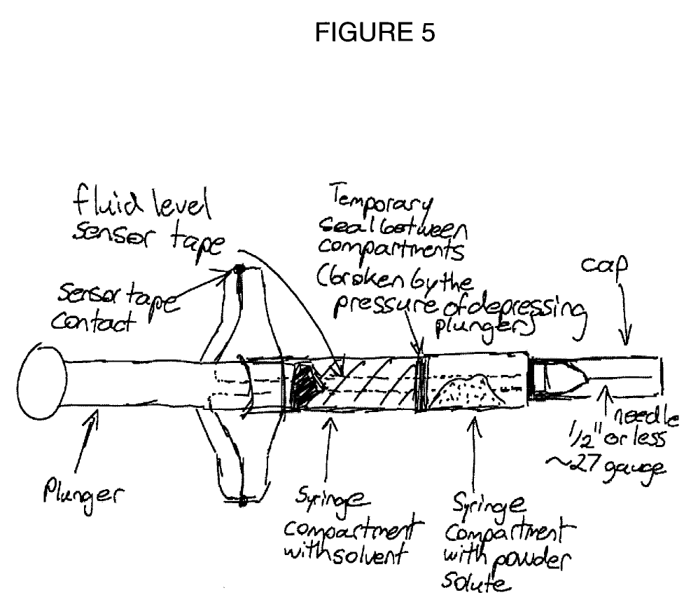

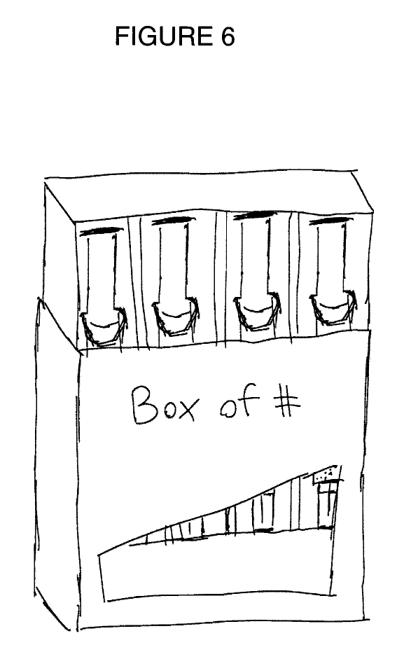

The infusate container(s), such as cartridges/vials, may be available, for example, in boxes (see FIG. 6). The infusate container(s) may be covered in opaque material, such as plastic lined foil. The infusate container, such as a cartridge or a vial, may include a syringe with a needle and a preattached cap and/or a preinserted plunger. The infusate container may be labeled, and lined with a tape, which may be a liquid level sensor tape, such as eTape by Milone Technologies. The infusate container, such as a cartridge or a vial, may include electrical contacts on its exterior surface, which may allow the pump to interface with the liquid level sensor tape. The infusate container may have at least two compartments. One of such compartments may contain a sterile solvent solution, which may be, for example, a sodium chloride solution in water. The concentration of the sodium chloride solution may be, for example, 0.9% (w/v). Preferably, the sterile solution compartment contains an amount of the sterile solution, which is sufficient to produce a nearly saturated solution when mixed with the content of the other compartment(s). The other compartment (the active agent compartment) may contain a sterile thyroxine and/or excipients/stabilizers, which may be in a lyophilized powder form. In some embodiments, the absence of excipients may be preferred. Yet in some embodiments, one or more excipients may be used. Such excepients may include one or more of the following: dextrose, mannitol, tribasic sodium phosphate, anhydrous and sodium hydroxide, hydrochloric acid, glycerin, dibasic sodium phosphate, m-cresol distilled, zinc, water, sodium chloride, diiodotyrosine, iodoquinone, iodide salts, such as potassium iodide. Additives in the active agent compartment may include, for example, T3 antibodies, T4 analogue antibodies, T3 analogue antibodies and/or fragments thereof. Such antibodies/antibody fragments additives may be coated/fixed onto the interior surface of the container, and/ or may be incorporated in the container on a membrane, such as a cellulose or cellophane semipermiable membrane, such that the antibodies/antibody fragments additives may not pass through the container's nozzle/opening. The compartments may separated by a temporary seal, which may broken by depressing the plunger in case of the syringe based container. Breaking the seal may allow for mixing the contents of the two compartments together thereby forming the infusate composition. After expelling air from the container's syringe, the infusate container may be ready to be inserted into the pump. A connector of the container within the pump assembly, may be equipped with an air escape valve or air eliminating membrane, allowing the pump to perform mixing of the compartments and expulsion of air, prior to initiating infusion, such that the container needs only be inserted and the contents of its compartments will only mixed upon initiating the infusion, in order to minimize accelerated thyroxine degradation once in a solution. In some embodiments, the container may include a stirrer, such as a magnetic stir bar, to facilitate stirring by a set of electromagnets within the container's socket of the pump assembly. FIG. 5 schematically illustrates a syringe based container.

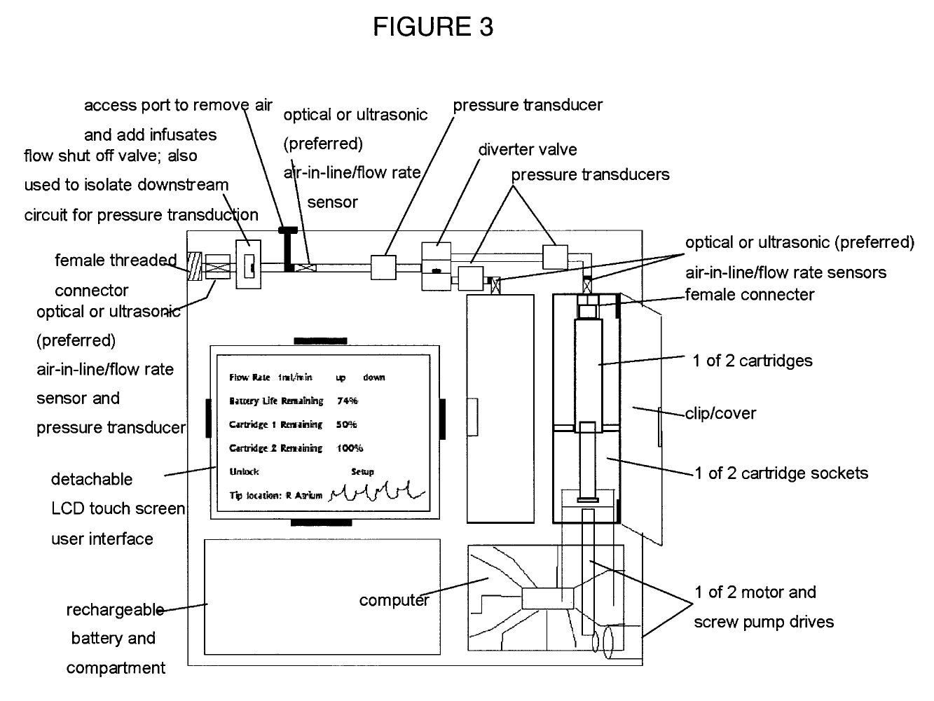

FIG. 3 schematically illustrates the following components of the external infusion system: a) a screw motor pump, b) a port (compartment) for an infusate container (cartridge) in the pump assembly; c) an attachment tool (cover) for holding the infusate container (cartridge) in the port (compartment) b); d) a diverter valve of the Y shaped conduit; e) a filter on the conduit in proximity of the catheter interface; f) a catheter interface port (catheter connector); g) a display (detachable touch screen display); h) a computer; i) a battery compartment.

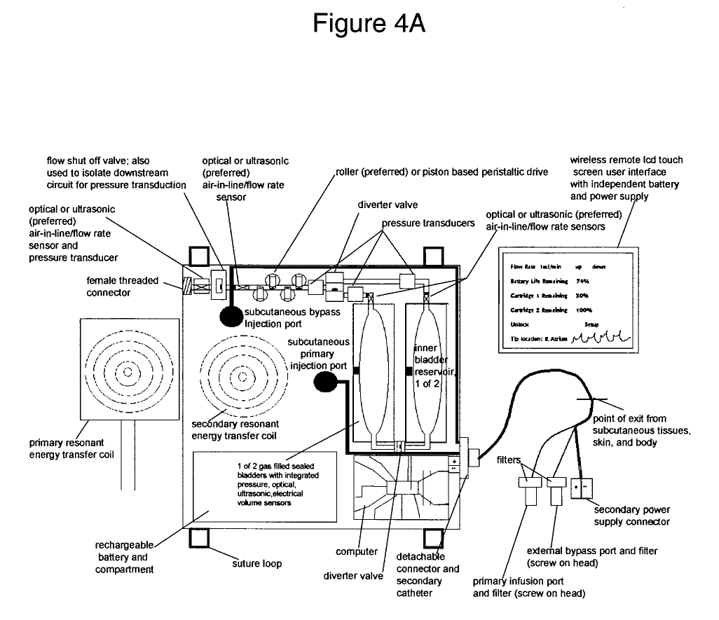

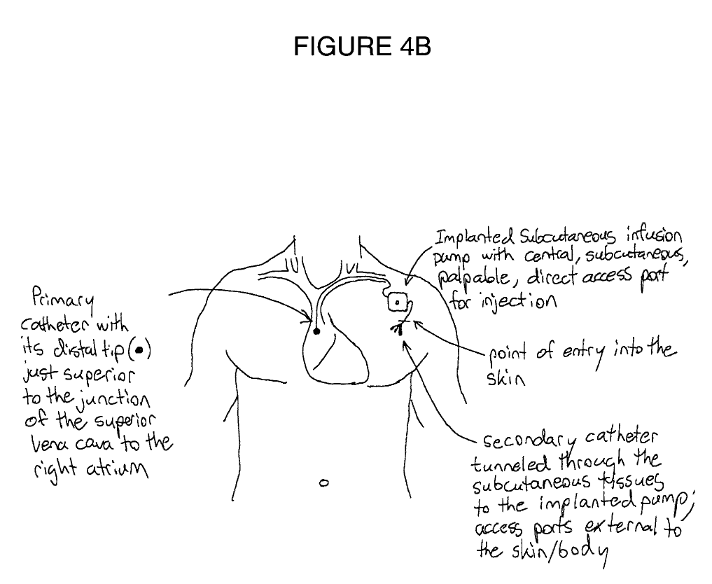

An example of an implantable infusion system may be based on Medtronic's SynchroMed II Programmable Drug Infusion System for chronic pain with modifications/adjustments made with respect to the pump, the catheter's length, design and placement, as well as in the number of phases in the cartridge syringe. The most significant difference with respect to the Medtronic's system may be that the infusate comprises a hormonal active agent that consists essentially of T4. The implantable infusion system and its components is schematically depicted in FIG. 4a.

The catheter of the infusions system may include a sterile, plastic tube, such as a polyurethane or silicone tube. The silicone tube may be preferred. The catheter may include a single proximal port, a lumen, which may be sufficiently narrow to minimize dead space without obstructing the infusate flow, and a single distal drug eluting tip. The catheter may have a length available up to 1.5 meters or up to 1 meter. The catheter may be provided in a sterile packaging, which may also include devices required for insertion by cannula-over-needle or Seldinger technique. Such devises may include needle, trocar, syringe and/or guidewire. The catheter may be connected to a pump assembly under the skin in or below the subcutaneous tissue. The catheter may be tunneled through the subcutaneous tissue to the site of entry into a blood vessel, such as a vein. The tunneling of the catheter may be done in a manner similar to a Hickman line. Upon the completion of the tunneling, the catheter may enter a blood vessel, which may be a vein, such as an antecubital vein, a femoral vein, a jugular vein, a subclavian vein or a brachiocephalic vein. Entering the subclavian vein may be preferred. Then the catheter may be advanced with its drug eluting tip being placed in one of the following final positions: 1. within the vein distal to the site of entry, 2. just superior to the junction of the superior vena cava to the right atrium, 3. just inferior to the junction of the inferior vena cava to the right atrium, 4. marginally within the right atrium. Positioning of the catheter and its distal tip may be confirmed with X-ray as well as by pressure transduction and analysis of the pressure waveform of the environment, where the catheter's tip is situated. The proximal tip of the catheter may be attached to the pump assembly beneath the skin of the patient.

The modified pump assembly may include a pump, which may be, for example, a battery powered, variable flow rate pump. The pump per se may be peristaltic, piston or roller driven. Preferably, the pump assembly is sterile and hermetically sealed. The pump may be contained in a case. Such a case may be made of a plastic or a metal, such as titanium. The pump assembly may be located under the skin of the patient. The pump assembly may be positioned, for example, in or under the subcutaneous tissue, but overlying the fascia. FIG. 4b schematically illustrates location of the implantable infusion system on a human body. The pump assembly may be equipped with a secondary resonant power transfer coil to supply power to the battery and the pump per se. The pump assembly may include a switch, which may be integrated into the circuit from the secondary power coil to the battery, preventing the coil from discharging the battery when not being used to recharge the battery. The pump assembly may include a primary resonant power transfer coil, which may be supplied in an unshielded casing. The primary resonant power transfer coil may draw its power from a transformer which may draw its power from an electrical outlet.

The pump assembly may include one or more two compartment containers or reservoirs. Such container(s) may be made of, for example, a plastic, such as silicone, ceramic or metallic material. In many embodiments, the two compartments of the container may form a single, unitary piece. The first (outer) compartment of the two compartment container may contain a gas sealed outer bladder, which may exert pressure in an elastic manner on the second (inner) compartment, which may be contained within the first. The second (inner) compartment may be an expandable (bladder) reservoir. The second (inner) compartment may contain inside the infusate and/or maintenance solution. The two compartments may be separated by an elastic wall. The second compartment may have an input and output conduits, which may comprise, for example, plastic or metal tubing. The inner wall of the outer compartment, which is the inner bladder reservoir, may be lined with two separated electrical contacts, as part of an electrical circuit, which may function to indicate emptying of the second compartment. The second compartment may be filled with the infusate and/or maintenance solution through a secondary catheter. During the operation of the system, the infusate and/or maintenance solution may be provided to the primary catheter from the inner compartment of the two compartment container/reservoir through the conduit of the implantable system.

The input ports of the pump assembly may be connected proximally (proximal meaning near to the site of the infusate injection, and distal meaning near to the connector for the proximal end of the primary catheter) to a diverter valve. The diverter valve may be connected via a conduit, which may be made, for example, of plastic or metal, to an injectable port, which may be slightly raised on the surface of the pump assembly, which may underlie the skin.

The diverter valve may be also connected to a secondary catheter, which may be a detachable and replaceable catheter. The detachable and replaceable secondary catheter may allow using the system without this catheter or allowing for an easy replacement of the catheter. The secondary catheter may contain a) at least two lumens, one of which leading to a diverter valve with leads to one or more two compartment containers/reservoirs and the other may be bypassing the two compartment container/reservoirs and the pump drive for infusion of other chemicals; and b) electrical conduits, which may be, for example, copper, or other metal wires, for a secondary means of electrical power transfer if needed. The secondary catheter may be tunneled through the subcutaneous tissues and may exit the skin, where its lumen(s) may be attached to one or more infusion ports, through which the infusate and/or maintenance solution may be supplied to the system. Two lumens of the secondary catheter may be connected to a single infusion port or to two separate infusion ports. The electrical conduits of the secondary catheter may be connected to the external power supply, if necessary. The external electrical contacts terminating the electrical conduit of the secondary catheter may be replaced if necessary.

The infusion port, to which the lumen of the secondary catheter may be attached, may include one or more filters. The infusion port may include a screw on head for the lumen attachment. In case of more than one two-compartment containers/reservoirs, the pump may automatically switch its infusate supply between them, using a diverter valve, as one of becomes depleted, allowing for replacement of the infusate without interrupting the infusion. The pump assembly may include one or more of the following: a resistance sensor in the motor, one or more pressure transducers along the infusion conduit, a pressure sensor, which may attached to the outer (first) compartment of the two-compartment container/reservoir, one or more optical and/or ultrasonic sensors, which may signal emptying of a bladder reservoir, directing the computer to provide a signal, such as an audible signal, and change the position of the diverter valve.

The implantable pump assembly may include an external remote display, which may be a lcd touch display. The display may have a wireless transmitter-receiver which may allow it to communicate with the system's computer. The display may have an independent power supply. The display may act as a user's interface with the system. The display may be used, for example, to operate the system, which may involve performing calibration, defining preferences and flow rate, as well as to display the system's and/or patient's parameters and information, such as pressure waveform, pump diagnostic information, amount of the infusate remaining in each cartridge, and displaying battery life remaining

The system may include a computer, which may be a computer built into the pump assembly. The computer may be used, for example, control pump activity based on user settings; control the diverter valve; monitor the amount of the infusate remaining; monitor, record and/or analyze the pressure wave form; monitor for the presence of air within the infusion circuit, monitor for infusion circuit obstruction, monitor the battery life remaining; communicate with the display and/or to produce signals, which may, for example, signal the battery depletion and the infusate's container/reservoir depletion, presence of air within the infusion circuit, and the location of the catheter's distal tip. The signals produced by the computer may be audible signals. For producing the audible signals, the system may include a speaker, which may integrated with the computer.

In many embodiments, the pump per se, the computer and/or the battery compartments may be thermally isolated within the pump assembly from the infusate containing container(s), to minimize heat transfer into the container(s). The thermal isolation may involve, for example, separating the pump per se, the computer and/or the battery compartments from the infusate containing container(s), such as two compartment container(s)/reservoir(s), with a vacuum sealed, plastic lined layer.

The sequence of devices of the implantable system (some of which are illustrated in FIG. 4a) from proximal to distal may be as follows: 1) a detachable secondary catheter, which may be used for access via injection bypassing the pump drive and reservoirs, secondary power supply, access via injection to the proximal end of the infusion conduit leading to the proximal diverter valve, which also may act as a shut-off valve, and is also supplied by the primary subcutaneous injectable plastic port located on the central surface of the pump; 2) two two-compartment bladder containers/reservoirs, which may be equipped with pressure sensors; 3.) an air-in-line/flow rate sensor, which may be, for example, an optical or an ultrasonic sensor, 4) one or more pressure transducers, 5) a distal diverter valve, which may be used as a shut-off valve as well , 6) a pressure transducer, 7) a roller based or piston based peristaltic pump drive; 8) an air-in-line/flow rate sensor, which may be, for example, an optical or an ultrasonic sensor, 9) two conduit branches: a. one leading to the injectable subcutaneous plastic bypass port on the surface of the pump, the other leading to the secondary catheter, 10) a flow shut-off valve, 11. a combined air-in-line/flow rate sensor pressure transducer, 12. a primary catheter connector, which may be without a filter. The catheter connector may be a female threaded connector.

The pump assembly may have suture loops affixed to its perimeter, to allow it, if needed to be sutured in place, under the skin.

External infusate or maintenance solution containers, such as cartridges or vials, may be the same as the infusate containers used in the external infusion system. The infusate from the external container may be inserted to the two compartment containers/reservoirs either directly or through the secondary catheter. In the implantable infusion system, it may be preferred to use such external infusate solution container(s), which have the same volume as two-compartment container(s)/reservoir(s) of the pump assembly.

### REFERENCES

Brunova J, et al. S Afr Med J. 2003 Jul; 93(7): 515-6.

Ross IL, et al. S Afr Med J. 2003 Jul; 93(7): 529-31.

Tigas S, et al. Thyroid. 2000 Dec; 10(12): 1107-11.

Dale J, et al. Clin Endocrinol (Oxf). 2001 Aug; 55(2): 233-9.

Pears J, et al. Scott Med J. 1990 Dec; 35(6): 180-2.

Saravanan P, et al. Clin Endocrinol (Oxf). 2002 Nov; 57(5): 577-85.

Wojewoda B, et al. Rocz Akad Med Bialymst. 2005; 50 Suppl 1: 133-6.

Cohen N, et al. Gynecol Endocrinol. 2010 Jun 14. PMID: 20540671.

Kronenberg H, et al.. 2008. Williams Textbook of Endocrinology. 11th ed. Saunders Elsevier. Philadelphia.

Cummings C, et al. 2010. Otolaryngology: Head & Neck Surgery. 5th ed. Mosby Elsevier. Philadelphia.

Katzung B. 1998. Basic and Clinical Pharmacology. 7th ed. 1998. Appleton & Lang. New York.

Restorative Medicine. 2010. Supraphysiological cyclic dosing of sustained release T3 in order to reset low basal body temperature.

http://www.restorativemedicine.com/articles/ supraphysiological-cyclic-dosing-of-sustained-release-t3-in-order-to-reset-low-basal-body-temperature/Dryden GL, et al. Gen Comp Endocrinol. 1969 Jun; 12(3): 536-40.

Chopra IJ.. J Clin Invest. 1976 Jul; 58(1): 32-40.

Knudsen N, et al. J Clin Endocrinol Metab. 2005 Jul; 90(7): 4019-24. Epub 2005 May 3.

Michalaki MA, et al. Thyroid. 2006 Jan; 16(1): 73-8.

Kim BJ, et al. Clin Endocrinol (Oxf). 2009 Jan; 70(1): 152-60. Epub 2008 May 20.

Chomard P, et al. Hum Nutr Clin Nutr. 1985 Sep; 39(5): 371-8.

Valizadeh M, et al. Endocr Res. 2009; 34(3): 80-9.

Clyde PW, et al. JAMA. 2003 Dec 10; 290(22): 2952-8.

Escobar-Morreale HF, et al.J Clin Endocrinol Metab. 2005 Aug; 90(8): 4946-54. Epub 2005 May 31.

Siegmund W, et al. Clin Endocrinol (Oxf). 2004 Jun; 60(6): 750-7.

Regalbuto C, et al. Thyroid. 2007 Apr; 17(4): 323-31.

Rangan S, et al. Endocrine Abstracts (2006) 12 P15.

Bauhofer G, et al. Acta Med Austriaca. 1976; 3(2): 54-8.

Grebe SK, et al. J Clin Endocrinol Metab. 1997 Mar; 82(3): 870-5.

Katzung B, et al. Basic & Clinical Pharmacology, Eleventh Edition. 2009. The McGraw-Hill Companies. New York.

The present invention can be illustrated in more detail by the following example, however, it should be understood that the present invention is not limited thereto.

### EXAMPLE

The following case report further can demonstrate why reducing fluctuation (F) in delivery of thyroid hormone may be desirable. The subject (Sam), of this case report, was treated with radioactive iodine ablation with a relatively low dose. Prior to diagnosis of Grave's disease, Sam was never known to have free T4 levels above median, and had always maintained the same weight +/−3 lbs beyond the age of 18.

Subsequent to treatment, Sam was found to have elevated TSH and low free T4 concentrations and was placed on 0.125 mg OD of T4; following several months of treatment, Sam began to experience severe nasal congestion and dysphoria, and a change in weight by a mean of 5 lbs, which was persistent. Noting TSH to be elevated, over a period of many months, Sam titrated (with a minimum of 6 weeks at any given dose) to doses ranging from 0.1 mg OD to 0.25 mg OD (experiencing palpitations, anxiety, stiffness, chest tightness, insomnia, sclerotic skin changes, sweating and heat intolerance, with free T4 levels reaching 28 pmol/L, and eventually suppressing TSH); Sam returned to the initial dose of 0.125 mg OD, where he remained with continued symptoms of nasal congestion and dysphoria, with a brief reprieve only to regress. Noting TSH to be elevated yet again, Sam increased his dose to 0.15 mg OD, with no improvement in his symptoms.

Over the next several years, attempting to ameliorate symptoms, Sam attempted to treat the hypothyroid state with the following means: 0.175 mg of T4 OD, resulting in palpitations, anxiety, stiffness, chest tightness, insomnia, sclerotic skin changes, sweating and heat intolerance within 6 months of initiating therapy. The severity of the symptoms required Sam to return to return to 0.15 mg OD of T4 within 1 week of experiencing the symptoms—remaining at that dose for 6 weeks. Biochemistry was actually analyzed during this period and yielded surprising results: TSH remained normal for 5 months, and during the 6th month (far beyond what would have been expected to be steady state) became undetectable. More surprising was that free T4 rose, and free T3 became elevated beyond the normal range, in the 6th month, even though TSH had become suppressed and therefore any endogenous production that may have been present would have been reduced/ceased. This was followed by an attempt to treat with: 0.125 mg of T4 with 0.005 mg of T3 OD, resulting in palpitations, anxiety, stiffness, chest tightness, insomnia, sclerotic skin changes, sweating and heat intolerance within 3 weeks of initiating therapy. The severity of the symptoms required Sam to return to 0.15 mg OD of T4 within 2 days of experiencing these symptoms—remaining at that dose for 6 weeks with resolution of the adrenergic symptoms. This was followed by an attempt to treat with: 0.035 mg of T3 OD, resulting in palpitations, anxiety, stiffness, chest tightness, insomnia, sclerotic skin changes, sweating and heat intolerance within 1 week of initiating therapy. The severity of the symptoms required Sam to return to 0.15 mg OD of T4 within 1 day of experiencing these symptoms—remaining at that dose for 6 weeks with resolution of the adrenergic symptoms. This was followed by an attempt to treat with: 0.005 mg of T3 q3:25hours, resulting in palpitations, anxiety, stiffness, chest tightness, insomnia, sclerotic skin changes, sweating and heat intolerance within 1 week of initiating therapy. The severity of the symptoms required Sam to return to 0.15 mg OD of T4 within 1 day of experiencing symptoms—remaining at that dose for many years, with continued nasal congestion and dysphoria.

After many years had passed, Sam suddenly experienced the following: rapid weight gain (of 25 lbs), rapid body fat increase (from 22% to 42%, by electrical impedance, from a lean mass of 117 lbs to a lean mass of 101 lbs) increase in abdominal girth, facial changes greater resembling acromegaly, hairline recession, insomnia, anorexia, night sweats, cold intolerance, weakness, loss of muscle mass and definition, and the sensation of weight loss.

The processes seemed to slow, and the symptoms seemed to settle over many months, but persisted until Sam attempted the following treatment: 0.088 mg of T4 q8hours, resulting in resolution of the previous symptoms within one week, and onset of dysphoria, nasal congestion, and mild to moderate palpitations, anxiety, stiffness, chest tightness, insomnia, sclerotic skin changes, sweating and heat intolerance for a period of three weeks. Then the symptoms all stopped. Surprisingly, the dysphoria and nasal congestion, that had plagued Sam for years, disappeared. Sam continued this dose for several months. T4 concentrations were higher than normal range and TSH was suppressed, however Sam experienced many benefits beyond once daily dosing, including increased subjective sensation of fat loss (less palpable subcutaneous fat and increased skin turgor) and subjective sensation of increased strength, until Sam attempted to treat with the following: 0.075 mg of T4 q8hours, resulting in weight loss (2-3 pounds), increased sensation of fat gain (more palpable subcutaneous fat and decreased skin turgor), dysphoria, nasal congestion, palpitations, insomnia, anorexia, night sweats, sclerotic skin changes, cold sweats, cold intolerance, weakness and the sensation of weight loss which continued beyond three weeks, and so Sam attempted to treat with the following: 0.1 mg of T4 q8hours, resulting in weight gain (2-3 pounds), dysphoria, nasal congestion, and mild to moderate palpitations, anxiety, stiffness, chest tightness, insomnia, sclerotic skin changes, sweating and heat intolerance for a period of three weeks. Then the symptoms improved significantly, but did not resolve completely. Dysphoria and nasal congestion were improved over that which was present with once daily dosing, but still remained present, so Sam attempted to treat with the following: 0.075 mg of T4 q6hours, resulting in dysphoria, nasal congestion, and mild to moderate palpitations, anxiety, stiffness, chest tightness, insomnia, sweating and heat intolerance for 2 days, then the symptoms stopped, until Sam changed treatment to: 0.05 mg of T4 q4hours, resulting in dysphoria, nasal congestion, and mild palpitations, anxiety, stiffness, chest tightness, insomnia, sweating and heat intolerance for 2 days, then the symptoms stopped, with an even greater sense of well being than had been experienced on previous treatment regimes, but Sam further increased the dose to: 0.05 mg of T4 q3hrous, resulting in dysphoria, nasal congestion, and mild palpitations, anxiety, stiffness, chest tightness, insomnia, sclerotic skin changes, sweating and heat intolerance for 3 weeks, then the symptoms stopped. Sam remained on this dose for a period of approximately 3 years. No tachycardia (except during significant exertion—felt to be more than prior to treatment with T4), tachypnea (except during significant exertion—felt to be more than prior to treatment with T4), hypertension was observed in that time; EKG (normal sinus rhythm at 71 beats per minute), chest X-ray and abdominal/pelvic ultrasound at 2.7 years were all normal; blood analysis done at that time yielded normal blood glucose, exceptional lipid profile, no biochemical imbalances, no hematologic abnormalities (however, reference ranges for white blood cell count, and mean corpuscular volume were dramatically different than those commonly known, the accuracy of these reference ranges was never determined, and the testing was never repeated), normal urinalysis, normal parathyroid hormone values, however serum calcium was slightly above the upper limit of normal. Free T4 concentration gradually decreased (by 15%), but remained far above normal and TSH remained suppressed. Weight loss of 40 lbs (with a resulting body fat of 26% by electrical impedance, and lean mass of 100 lbs.) over the first 15 months occurred followed by weight gain of 15 lbs over the next 15 months (with a resulting body fat of 28% by electrical impedance, and lean mass of 108 lbs.). A reduced incidence and duration of common colds was noted, while at steady state over the 3 year period. On one occasion, when the expiry date of T4 administered was several months nearer to the time of administration, than that of T4 typically administered by Sam, within one day, onset of dysphoria, nasal congestion, palpitations, insomnia, anorexia, night sweats, cold sweats, cold intolerance, weakness and the sensation of weight loss occurred; the symptoms resolved within three days. Similar symptoms ensued if Sam was more than two hours late administering a dose; symptoms lasted only several hours. Conversely, if Sam was more than two hours early in administering a dose, symptoms of dysphoria, nasal congestion, and mild to moderate palpitations, anxiety, stiffness, chest tightness, insomnia, sweating and heat intolerance occurred, lasting only several hours. Due to concerns regarding safety, primarily due to bone mineral density loss, dosing was reduced (as Sam was instructed to do throughout the course of treatment where treatment exceeded recommended dosing guidelines) to: 0.05 mg of T4 q4hours, resulting in a 2 to 3 lbs weight loss and, surprisingly, a free T4 rise (22%) within the first 2 weeks, and dysphoria, nasal congestion, palpitations, insomnia, anorexia, night sweats, sclerotic skin changes, cold sweats, cold intolerance, weakness and the sensation of weight loss ensued for a period of 8 weeks and then resolved. Sam was urged to further reduce total dosing to within recommended prescribing guidelines; it should be noted that the author was not the prescribing physician.

This case report demonstrates a benefit for delivery of T4 with a fluctuation (F) of less than that caused by the oral administration of a conventional form of T4 (Eltroxin or Synthroid) at a dose of 100 ug over an eight hour period, preferably less than that caused by oral administration of a conventional form of T4 at a dose of 88 ug over an eight hour period, with better outcome (reduced severity of adverse symptoms) the more closely F approaches zero. The case report also demonstrates that T4 may be used to increase/maintain lean mass, and to decrease body fat.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention. All the publications, patent applications and patents cited in this specification are incorporated herein by reference in their entirety.

What is claimed is:

1. An infusion system comprising (a) a container containing an infusate consisting essentially of thyroxine and (b) a driving member configured to drive said infusate from the container in a continuous manner.

2. The infusion system of claim 1, wherein the driving member is a pump.

3. The infusion system of claim 1, further comprising an implantable catheter in fluidic connection with said container.

4. The infusion system of claim 1, wherein said container is a replaceable cartridge and wherein the system further comprises a port configured to receive said cartridge.

5. The infusion system of claim 1, wherein the thyroxine comprises 3,5,3′,5′ tetraiodo-L-thyronine.

6. The infusion system of claim 1, wherein the thyroxine comprises a pharmaceutically acceptable salt of 3,5,3′,5′ tetraiodo-L-thyronine.

7. The infusion system of claim 1, wherein the thyroxine comprises a prodrug of 3,5,3′,5′ tetraiodo-L-thyronine.

8. The infusion system of claim 1, wherein the thyroxine comprises a pharmaceutically acceptable salt of a prodrug of 3,5,3′,5′ tetraiodo-L-thyronine.

9. A method of treating of a thyroid disorder comprising administering to a subject in need thereof a hormone active agent consisting essentially of thyroxine via the infusion system of claim 1.

10. A plurality of thyroxine oral forms, wherein each of said forms comprises a hormonal active agent that consists essentially of thyroxine and wherein an incremental difference between said forms in the amount of the thyroxine present is no greater than 0.0000000125179946172623 moles.

11. The plurality of thyroxine oral forms according to claim 10, wherein the incremental difference is no greater than 0.00000000625899730863116 moles.

12. The plurality of thyroxine oral forms according to claim 10, wherein the thyroxine comprises 3,5,3′,5′ tetraiodo-L-thyronine.

13. The plurality of thyroxine oral forms according to claim 10, wherein the thyroxine comprises a pharmaceutically acceptable salt of 3,5,3′,5′ tetraiodo-L-thyronine.

14. The plurality of thyroxine oral forms according to claim 10, wherein the thyroxine comprises a prodrug of 3,5,3′,5′ tetraiodo-L-thyronine.

15. The plurality of thyroxine oral forms according to claim 10, wherein the thyroxine comprises a pharmaceutically acceptable salt of a prodrug of 3,5,3′,5′ tetraiodo-L-thyronine.

16. A method of treating of a thyroid disorder comprising administering to a subject in need thereof a thyroid hormone active agent consisting essentially of thyroxine via one or more of the oral dosage forms of claim 10.

17. An oral dosage form comprising a controlled release matrix that contains a hormone active agent consisting essentially of thyroxine.

18. The oral dosage form of claim 17, wherein a characteristic release time of said matrix is greater than 3 hours.

19. The oral dosage form of claim 17, wherein the thyroxine comprises 3,5,3′,5′ tetraiodo-L-thyronine.

20. The oral dosage form of claim 17, wherein the thyroxine comprises a pharmaceutically acceptable salt of 3,5,3′,5′ tetraiodo-L-thyronine.

21. The oral dosage form of claim 17, wherein the thyroxine comprises a prodrug of 3,5,3′,5′ tetraiodo-L-thyronine.

22. The oral dosage form of claim 17, wherein the thyroxine comprises a pharmaceutically acceptable salt of a prodrug of 3,5,3′,5′ tetraiodo-L-thyronine.

23. A method of treating of a thyroid disorder comprising administering to a subject in need thereof a thyroid hormone active agent consisting essentially of thyroxine via the oral dosage form of claim 17.
